(19) United States
(12) Patent Application Publication
(10) Pub. No.: US 2003/0077761 A1
(43) Pub. Date: Apr. 24, 2003

(54) Methods

(76) Inventors: Vendela Parrow, Uppsala (SE); Linda Rosengren, Uppsala (SE)

Correspondence Address: JANIS K. FRASER, PH.D., Fish & Richardson P.C., 225 Franklin Street, Boston, MA, 02110-2804, US

(21) Appl. No.: 10/161,088

(22) Filed: May 31, 2002

(30) Foreign Application Priority Data
Jun. 1, 2001 (SE) 0101934-8

(51) Int. Cl. C12Q 1/68; C12P 19/34

(52) U.S. Cl. 435/91.2; 435/6

(57) ABSTRACT

The invention provides a quantitative hybridization assay for the analysis of mRNA in a target nucleic acid sample. The method comprising the steps of (i) immobilizing the target nucleic acid sample on a solid support; (ii) contacting a labeled antisense probe to a first portion of the said target nucleic acid sample, and a labeled sense probe to a second portion of the said target nucleic acid sample; (iii) detecting and quantitating the signals generated from hybridized antisense probe and hybridized sense probe; and (iv) determining the value represented by the antisense probe signal minus the sense probe signal, said value being proportional to the amount of mRNA in the target nucleic acid sample.

### CROSS REFERENCE TO RELATED APPLICATIONS

This application claims priority from Swedish Patent Application No. 0101934-8, filed Jun. 1, 2001. This entire content of the prior application is incorporated herein by reference.

### TECHNICAL FIELD

The present invention relates to quantitative hybridization assays for the analysis of mRNA in nucleic acid samples.

### BACKGROUND ART

Induction of specific RNA expression is an important marker of biological activity of a drug or chemical compound. The quantitation of RNA in a sample is necessary in order to determine whether an agent is capable of inducing RNA expression. Various RNA quantitation methods (RNA hybridization assays) are known in the art. However, the currently used methods normally require the isolation and purification of RNA, additional cDNA synthesis, the use of special equipment, and/or the use of sets of a large number (20 or more) species-specific oligonucleotides (branched DNA assays).

It is often desirable to determine the RNA-inducing effect of various agents in samples from more than one animal species. According to standard methods, the RNA quantitation assay can be carried out in monolayers of cells or tissue slices transferred to microplates. The agents to be tested are then added to the wells of the plate, often by use of pipetting robots. Microplate readers are then used for detecting fluorescence or luminescence, indicating that RNA in the sample has hybridized to a labeled probe.

Consequently, there is a need for simple and reliable RNA quantitation methods that do not require RNA purification or the use of special equipment, which are compatible with the standard microplate format, and in which the same probes could be used for hybridization to RNA samples from more than one animal species.

It would be particularly useful to be able to correlate a determined RNA expression level to an internal standard, thus eliminating variations resulting from the use of different amounts of starting material in different assays.

### BRIEF DESCRIPTION OF THE DRAWINGS

FIG. 1 is a graph depicting the detection of incorporated biotin label in sense- and anti sense probes derived from the mouse IGF-I gene (LCPS=Luminescence Counts Per Second).

FIG. 2 is a graph depicting the quantitation of IGF-IB RNA in mouse hepatocytes after stimulation with various amounts of growth hormone (GH). The control (CTR) cells did not receive GH.

FIG. 3 is a graph depicting the quantitation of IGF-IB RNA in rat hepatocytes after stimulation with various amounts of growth hormone (GH). The control (CTR) cells did not receive GH.

### DISCLOSURE OF THE INVENTION

The present invention provides improved quantitative RNA hybridization assays. Advantageous features of the hybridization assays according to the invention include:

(a) the use of antisense- and sense probes having substantially the same length and specific activity, enabling for the easy determination of a value proportional to the amount of RNA, e.g., mRNA, of interest in a sample;

(b) the ability to use cell lysates directly in the hybridization assay without the need for previous purification of RNA; and

(c) the ability to use the same probes for the analysis of nucleic acid samples from several animal species, as the hybridization conditions are similar to standard conditions used for Northern and Southern blotting.

More specifically, the present invention provides a method for quantitation of a target nucleic acid in a sample, the method including: (i) immobilizing on a solid support a sample containing a target nucleic acid; (ii) under hybridization conditions, contacting a labeled antisense probe to a first portion of the sample containing the target nucleic acid and contacting a labeled sense probe to a second portion of the sample containing the target nucleic acid, wherein the antisense probe is complementary to all or a portion of the target nucleic acid, wherein the sense probe is identical to all or a portion of the target nucleic acid, and wherein the antisense and sense probes are substantially the same length and are capable of generating signals of substantially the same specific activity; (iii) detecting and quantitating signals generated from the antisense probe and the sense probe; and (iv) determining a value represented by the antisense probe signal minus the sense probe signal, the value being proportional to the amount of the target nucleic acid present in the sample.

The target nucleic acid can be an RNA, e.g., an mRNA. The sample containing the target nucleic acid can include the mRNA content of a cell or a cell population. For example, the sample can contain a cell lysate. The sample can be prepared, for example, by obtaining a total cell lysate from a cell culture.

In one embodiment, the antisense and sense probes are transcribed in vitro. In another example, the probes are made by solid phase synthesis methods. The probes can be RNA or DNA.

In one embodiment, the method additionally includes a step of removing unhybridized labeled probes prior to the detecting step (iii).

In one embodiment, the solid sunport can be a nylon membrane.

The probes can be labeled, for example, with a radioactive label or with a non-radioactive label, e.g., biotin.

In a further aspect, the invention includes a kit containing reagents for carrying out a method according to the invention. For example, the kit can include antisense and sense probes as described herein. The kit can also include a solid support and/or materials for generating a cell lysate and preparing the sample for analysis. The kit can optionally include instructions for use of the reagents according to a method described herein.

The invention will be further defined and described in more detail in the following sections:

Probes

The term “probe” denotes a single-stranded nucleic acid molecule of specific base sequence, which is used to detect the complementary base sequence by hybridization. In the present context, the term “antisense probe” denotes an RNA probe resulting from transcription of a part of the antisense (non-coding) strand of the DNA of choice. The antisense probe is consequently complementary to translatable mRNA transcribed from the said DNA, and capable of hybridizing to such mRNA. The term “sense probe” denotes an RNA probe resulting from transcription of a part of the sense DNA strand, i.e. the DNA strand used for synthesis of complementary mRNA. It will thus be understood that the sense probe will not be capable of significantly hybridizing to such mRNA.

According to the invention, the antisense and sense probes have essentially the same length, e.g., differing by no more than 100, 50, 25, 20, 15, 10, 5, 4, 3, 2, or nucleotides in length. The length of the probes will suitably range from approximately 100 to 1000 nucleotides in length.

Suitable sense- and antisense probes can be prepared from a DNA of interest by standard methods. One suitable way to prepare probes is to prepare a cDNA clone, comprising preferably 100-1000, e.g., around 300, nucleotides of the gene of interest, so that a promoter is ligated to either end of the cDNA, making it possible to in vitro transcribe the cDNA from both directions. The cDNA clone is linearized with suitable restriction enzymes so that the distance from the promoter to the restriction site is similar from either end of the cDNA. RNA probes (sense and antisense) can be prepared by in vitro transcription, for example using a commercially available transcription kit, such as a MAXIscript™ In Vitro Trancription Kit (Ambion Cat. No. 1308-1326). Common RNA polymerases used in in vitro transcription reactions are SP6, T7 and T3 polymerases, named for the bacteriophages from which they were cloned.

Label and Signal System

The term “labeled (sense or antisense) probe” refers to a probe that is capable of providing a detectable signal, either directly or through interaction with one or more additional members of a signal producing system. Labels that are directly detectable include radioactive and fluorescent labels. Labels that are detectable via a signal producing system include e.g. biotin, fluorescein, and low energy radioactivity. Biotinylated probes are known in the art for the sensitive colorimetric detection of target nucleic acid sequences on nitrocellulose filters, as shown in Leary et al. (1983) Proc. Natl. Acad. Sci. U.S.A. 80: 4045, and for in situ detection of target DNAs, as in Langer-Safer (1982) et al., Proc. Natl. Acad. Sci. U.S.A. 79: 4381.

In the examples provided below, the probes were labeled by cross-linking using BrightStar™ Psoralen-Biotin™ (Ambion Cat. No. 1480) according to the manufacturers recommendations. BrightStar™ Psoralen-Biotin™ consists of a tricyclic planar intercalating moiety, psoralen, which is covalently attached to biotin. This psoralen-biotin intercalates within the nucleic acid and is then covalently bound by brief irradiation with long-wave UV light.

According to the invention, the antisense and sense probes are labeled with substantially the same specific activity. The phrase “same specific activity” refers to the ability of the sense and antisense probes to generate the same intensity of signal relative to the amount of probe that has hybridized to the target nucleic acid sample. The phrase “same specific activity” also refers to the fact that the probes have essentially the same length and base composition, and that the number of labeled nucleotides are the same in both probes.

It is useful to check that the probes are labeled to the same specific activity. When the label is a radioactive one, its incorporation can be determined by scintillation counting of an aliquot of the purified probe. Incorporation of a non-radioactive label can be determined by spotting a serial dilution of antisense- and sense probes on a filter, followed by detection of fluorescence (if the label is fluorescein) or if the label is biotin, by detection system such as BrightStar™ BioDetect™ Nonisotopic Detection Kit (Ambion Cat. No. 1930). It is also useful that the antisense and sense probe concentration is determined accurately, e.g., by determining the optical density at 260 nm or by determining fluorescence according to standard methods, so that the same amount of each probe is used in each hybridization reaction.

Preparation and Immobilization of the Target Nucleic Acid Sample

The term “target nucleic acid sample” denotes a sample obtained from an animal tissue or cell culture, which sample comprises nucleic acids and wherein it is desirable to quantitate the amount of mRNA.

According to the present invention, the target nucleic acid sample is preferably prepared as a total lysate of a cell culture. Such a total cell lysate can be prepared according to known methods, see e.g. Kaabache, T. et al. (1995) Direct Solution Hybridization of Guanidine Thiocyanate Solubilized Cells for Quantitation of mRNAs in Hepatocytes. Anal. Biochem. 232: 225-230.

Normally, the cells are cultured to a suitable density and then lysed by addition of a suitable agent, such as guanidine thiocyanate. After lysis, aliquots of the cellular lysate are transferred to a suitable solid support. An example of a suitable solid support is a microplate-format nylon filter, for example “Nylon Membrane Filtermat”. The Filtermat (Perkin-Elmer; Product No. 1450-423) is a 96-position nylon membrane with a printed pattern, intended for use with a manifold for RNA/DNA hybridization assays. Typically, 5-10 μl of the lysate are spotted on a dry filter. Eight replica filters can be prepared in this manner. The lysate is then immobilized on the filter by UV-crosslinking, or by baking, according to standard methods.

Hybridization of the Probes to the Immobilized Target Sample

The term “hybridization conditions” denotes conditions sufficient to produce detectable hybridization between the RNA probe(s) and complementary nucleic acids comprised in the target nucleic acid sample. The skilled person will be able to determine suitable hybridization conditions from, for instance, well-known laboratory manuals.

In the Examples given below, the replica filters were hybridized at high stringency in a rolling bottle, using the commercially available ULTRAhyb™ Ultrasensitive Hybridization Buffer (Ambion Cat. No. 8670). After hybridization, the filters were washed at high stringency according to the ULTRAhyb™ protocol.

Detection and Quantitation of the Signal

The amount of probe that has hybridized to the target nucleic acid sample can be determined by standard methods. When the solid support is a membrane or filter having standard microplate format, the signal can conveniently be detected by using a microplate reader.

If the probes have been labeled with a radioactive label, the filters are dried, and hybridization can be quantified directly on the filters by autoradiography or phosphoimager analysis. Alternatively, a scintillation medium (such as MeltiLex™ solid scintillator (Perkin-Elmer; Product No. 1450-441/442) or a liquid scintillation medium) can be added to the filters, which can be sealed in plastic bags and counted in a microplate reader equipped for scintillation counting.

If the probes have been labeled with a non-radioactive label such as biotin, as in the Examples given below, the filters can be maintained in the rolling bottles (used during hybridization) during development of the signal. The signal can be detected using for instance the BrightStar™ BioDetect™ Nonisotopic Detection Kit. After addition of the luminescence substrate, the filters can be sealed in plastic, for example the commercially available Sample Bag (Perkin-Elmer; Product No. 1450-432) and transferred to filter holders. Luminescence is then determined in a microplate reader when the signal has reached maximum.

The signal generated from the antisense probe represents specific RNA present in the lysate, as well as the background hybridization to the total cellular lysate (part of which is derived from specific hybridization to DNA present in the lysate). In contrast, the signal generated from the sense probe represents only background hybridization, and is proportional to the amount of cellular material in the lysate. When the antisense- and sense probes are of the same length and same specific activity, the amount of signal from the sense-hybridization can be deducted from the antisense signal, resulting in a true estimate of the fold induction of a specific gene. Further, this results in a correction for possible variations in the amount of starting material, due to different amounts of cells or tissue prior to the lysis step.

Throughout this description the terms “standard protocols” and “standard procedures”, when used in the context of molecular biology techniques, are to be understood as protocols and procedures found in an ordinary laboratory manual such as: Current Protocols in Molecular Biology, editors F. Ausubel et al., John Wiley and Sons, Inc. 1994, or Sambrook, J., Fritsch, E. F. and Maniatis, T., Molecular Cloning: A laboratory manual, 2nd Ed., Cold Spring Harbor Laboratory Press, Cold Spring Harbor, N.Y. 1989.

Unless otherwise defined, all technical and scientific terms used herein have the same meaning as commonly understood by those of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In case of a conflict in terminology, the present specification will control. In addition, the described materials and methods are illustrative only and are not intended to be limiting.

The invention will now be described by way of examples, which are included of illustrative purposes only, and are not to be seen as limiting in any respect.

### EXAMPLES

### Example 1

### Preparation of Probes for Determination of IGF-IB RNA

A cDNA clone containing 60 nucleotides from exon 2, as well as 179 nucleotides from exon 3, of the mouse IGF-IB gene (GenBank Accession No. X04482; SEQ ID NOs: 1 and 2) were cloned into the pGEM®-4Z standard cloning vector (Promega; Cat. No. P2161). Linearization of the plasmid with EcoRI allowed transcription of an about 250 nucleotide long antisense probe using T7 polymerase (MAXIscript™ T7 kit; Ambion Cat. No 1308). Linearization with HindIII allowed transcription of a sense probe of similar length using SP6 polymerase (MAXIscript™ SP6 kit; Ambion Cat. No. 1312). The cDNA sequence of the transcribed IGF-IB fragment (lower case letters), as well as flanking plasmid sequences (upper case letters) is given below (SEQ ID NO: 3):

```
                          SP6→ EcoRI
                                  GAAT ACGAATTCGA GCATgcgggg
ctgagctggt ggatgctctt cagttcgtgt gtggaccgag
gggcttttac ttcaacaagc ccacaggcta tggctccagc
attcggaggg cacctcagac aggcattgtg gatgagtgtt
gcttccggag ctgtgatctg aggagactgg agatgtactg
tgccccactg aagcctacaa aagcagcccg ctctatccgt
gcccagcgcc acactgacat gcccaagact cagGCATGCA
                                                                                    Hi
AGCTTGTCTC CCTATAGTGAGT
ndIII
    ←T7
```

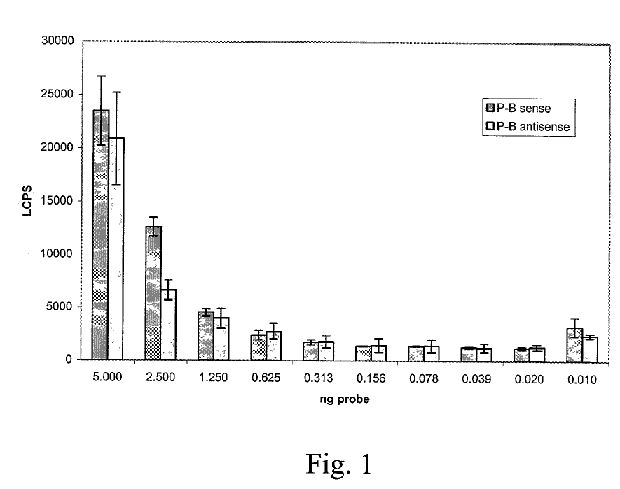

The probes were purified on a NICK-column (Amersham Pharmacia Biotech; Cat. No. 17-0855-02) and their concentrations were determined by optical density at 260 nm, as well as by ethidium bromide visualization according to standard methods. The same amount (0.5 μg) of antisense and sense probe was labeled by cross-linking with psoralen-biotin. Duplicate samples of serial dilutions were spotted onto filters and the amount of label was determined using the BrightStar™ BioDetect™ (Nonisotopic Detection) Kit (Ambion Cat. No. 1930) according to the manufacturers instruction. The results (FIG. 1) show that the amount of label was similar in corresponding amounts of antisense- and sense-probe.

### Example 2

### Determination of IGF-I RNA in Mouse Hepatocytes

Mouse hepatocytes were prepared by perfusion and plated in a 96-well microplate. The day after plating, the cells were subjected to serum starvation overnight. Thereafter the culture media was changed to fresh serum-free media and the cells were stimulated with a serial dilution of human growth hormone for 6 hours. The culture medium was removed and 100 μl lysis buffer (4.0-4.2 M guanidine thiocyanate and 0.1 M EDTA) was added to each well, which contained a monolayer of approximately 30,000 cells. The plate was immediately frozen to −70° C. and stored for three months.

After thawing the plates, lysis of the cells was achieved by pipetting the lysis buffer up and down several times using a pipetting robot. Aliquots (10 μl) of the lysate were transferred to nylon filters (Nylon Membrane Filtermat; Perkin-Elmer; Product No. 1450-423) using the same pipetting robot. Two replica filters were prepared. The lysate was immobilized on the filter by UV-crosslinking.

Hybridization was carried out at high stringency, using ULTRAhyb™ Ultrasensitive Hybridization Buffer (Ambion Cat. No. 8670) according to the manufacturers recommendations, at 68° C. in a rolling bottle. After pre-hybridization for one hour, antisense- and sense probes (0.5 ng/ml), prepared according to Example 1, were added to the first and second filter, respectively. After hybridization, the filters were washed at high stringency according to the ULTRAhyb™ protocol. The signals from hybridized probes were determined using the BrightStar™ BioDetect™ (Nonisotopic Detection) Kit (Ambion Cat. No. 1930) and a luminiscence reader.

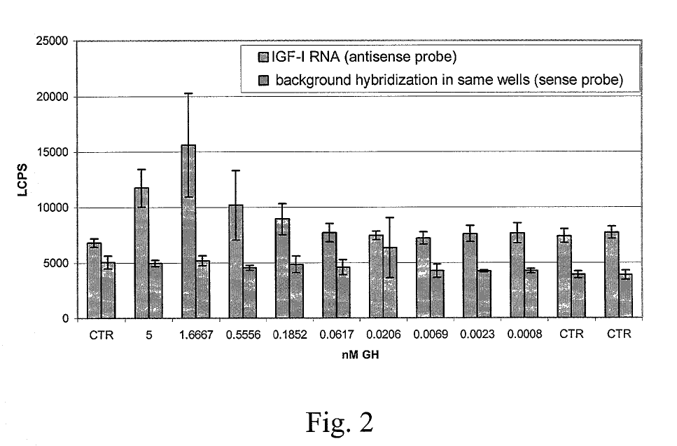

The results (FIG. 2) show a growth hormone-dependent induction of IGF-IB RNA, similar to what has been shown for rat hepatocytes (Tollet P. et al. (1990) Molecular Endocrinology 4: 1934-1942). The sense-hybridization showed that the cellular monolayer was even. When the background hybridization level obtained by the sense-probe was deducted from the antisense hybridization, RNA induction was shown to be similar (5 to 8-fold) to what has been reported from soluble hybridization studies in vivo and in vitro (Tollet P. et al., supra).

### Example 3

### Determination of IGF-I RNA in Rat Hepatocytes

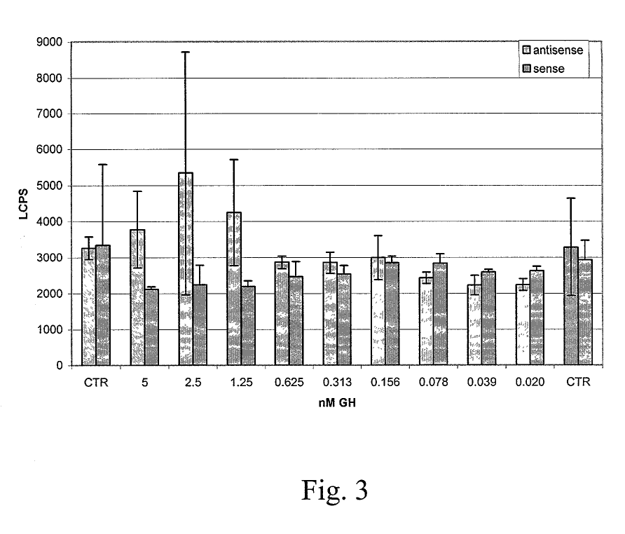

The experiment described in Example 2 was repeated with rat hepatocytes. As shown in FIG. 3, similar results were obtained as with mouse hepatocytes, indicating that the same pair of probes could be used for RNA quantification in samples from more than one animal species.

### Other Embodiments

It is to be understood that, while the invention has been described in conjunction with the detailed description thereof, the foregoing description is intended to illustrate and not limit the scope of the invention. Other aspects, advantages, and modifications of the invention are within the scope of the claims set forth below.

What is claimed is:

1. A method for quantitation of a target nucleic acid in a sample, the method comprising:
(i) immobilizing on a solid support a sample comprising a target nucleic acid;
(ii) under hybridization conditions, contacting a labeled antisense probe to a first portion of the sample comprising the target nucleic acid and contacting a labeled sense probe to a second portion of the sample comprising the target nucleic acid, wherein the antisense probe is complementary to all or a portion of the target nucleic acid, wherein the sense probe is identical to all or a portion of the target nucleic acid, and wherein the antisense and sense probes are substantially the same length and are capable of generating signals of substantially the same specific activity;
(iii) detecting and quantitating signals generated from the antisense probe and the sense probe; and
(iv) determining a value represented by the antisense probe signal minus the sense probe signal, the value being proportional to the amount of the target nucleic acid present in the sample.

2. The method of claim 1, wherein the target nucleic acid is an mRNA.

3. The method of claim 2, wherein the sample comprises a cell lysate.

4. The method of claim 1, wherein the antisense and sense probes are transcribed in vitro.

5. The method of claim 1, further comprising removing unhybridized labeled probes prior to the detecting step (iii).

6. The method of claim 1, wherein the sample comprising the target nucleic acid is prepared by obtaining a total cell lysate from a cell culture.

7. The method of claim 1, wherein the solid support is a nylon membrane.

8. The method of claim 1, wherein the probes are labeled with a non-radioactive label.

9. The method according to claim 8, wherein the non-radioactive label is biotin.

10. A kit comprising the antisense and sense probes of claim 1 and instructions for use.


SEQUENCE LISTING (3 sequences)

SEQ ID NO: 1 - DNA, 651 bp, Homo sapiens
  CDS (73)...(471)
acccactctgacctgctgtgtaaacgacccggacctaccaaaatgaccgcacctgcaataaagatacacatcatgtcgtcttcacacctcttctacctggcgctctgcttgctcaccttcaccagctccaccacagctggaccagagaccctttgcggggctgagctggtggatgctcttcagttcgtgtgtggaccgaggggcttttacttcaacaagcccacaggctatggctccagcattcggagggcacctcagacaggcattgtggatgagtgttgcttccggagctgtgatctgaggagactggagatgtactgtgccccactgaagcctacaaaagcagcccgctctatccgtgcccagcgccacactgacatgcccaagactcagaagtccccgtccctatcgacaaacaagaaaacgaagctgcaaaggagaaggaaaggaagtacatttgaagaacacaagtagaggaagtgcaggaaacaagacctacagaatgtaggaggagcctcccacggagcagaaaatgccacatcaccgcaggatcctttgctgcttgagcaacctgcaaaacatcgaaacacctaccaaataacaataataagtccaataacattacaaagatgggcatttcccccaatgaaa

SEQ ID NO: 2 - protein, 133 aa, Homo sapiens
MSSSHLFYLALCLLTFTSSTTAGPETLCGAELVDALQFVCGPRGFYFNKPTGYGSSIRRAPQTGIVDECCFRSCDLRRLEMYCAPLKPTKAARSIRAQRHTDMPKTQKSPSLSTNKKTKLQRRRKGSTFEEHK

SEQ ID NO: 3 - DNA, 286 bp, Homo sapiens
gaatacgaattcgagcatgcggggctgagctggtggatgctcttcagttcgtgtgtggaccgaggggcttttacttcaacaagcccacaggctatggctccagcattcggagggcacctcagacaggcattgtggatgagtgttgcttccggagctgtgatctgaggagactggagatgtactgtgccccactgaagcctacaaaagcagcccgctctatccgtgcccagcgccacactgacatgcccaagactcaggcatgcaagcttgtctccctatagtgagt